(19) United States
(12) Patent Application Publication
(10) Pub. No.: US 2026/0015750 A1
(43) Pub. Date: Jan. 15, 2026

(54) Electropolymerization For A Biological Sensor

(71) Applicant: Allez Health Inc., Carlsbad, CA (US)

(72) Inventors: Robert James Boock, Carlsbad, CA (US); Sai Kit Wu, Irvine, CA (US)

(73) Assignee: Allez Health Inc., Carlsbad, CA (US)

(21) Appl. No.: 19/262,578

(22) Filed: Jul. 8, 2025

Related U.S. Application Data
(60) Provisional application No. 63/669,568, filed on Jul. 10, 2024.

(51) Int. Cl. C25D 9/02; A61B 5/145; A61B 5/1486

(52) U.S. Cl. CPC C25D 9/02; A61B 5/14532; A61B 5/1486; A61B 2562/125

(57) ABSTRACT

A system includes a wire-holding fixture configured to be received in an EP station. The wire-holding fixture comprises terminals, and each terminal is electrically coupled to a respective working wire of a plurality of working wires. The working wires extend away from the wire-holding fixture. A set of EP stations is included, and each EP station is configured to receive the wire-holding fixture. A container is configured to hold an electropolymerization solution and positioned beneath the EP station such that the working wires is immersible. A board comprising a plurality of electrical contacts is included, and each electrical contact is configured to electrically engage a corresponding terminal. A controller is configured to apply a voltage to the working wires through the respective terminals and electrical contacts to initiate electropolymerization of the material from the electropolymerization solution to form a polymer layer on each working wire.

### RELATED APPLICATIONS

This application claims priority to U.S. Provisional Patent Application No. 63/669,568, filed on Jul. 10, 2024, and entitled “Automated Electropolymerization for a Biological Sensor”; the contents of which are incorporated by reference in full.

### BACKGROUND

Monitoring of glucose levels is critical for diabetes patients. Continuous glucose monitoring (CGM) sensors are a type of device in which glucose is measured from fluid sampled in an area just under the skin multiple times a day. CGM devices typically involve a small housing in which the electronics are located and which is adhered to the patient's skin to be worn for a period of time. A small needle within the device delivers the subcutaneous sensor which is often electrochemical.

The CGM sensor is typically temporarily adhered to the patient's skin with an adhesive pad, and the CGM sensor couples to a small housing in which electronics are located. The CGM sensor typically has a disposable applicator device that uses a small introducer needle to deliver the CGM sensor subcutaneously for the patient. Once the CGM sensor is in place, the applicator is discarded, and the electronics housing is attached to the sensor. Although the electronics housing is reusable and may be used for extended periods, the CGM sensor and applicator need to be replaced often, usually every few days.

Electrochemical glucose sensors operate by using electrodes which typically detect an amperometric signal caused by oxidation of enzymes during conversion of glucose to gluconolactone. The amperometric signal can then be correlated to a glucose concentration. The electrode typically includes an electrically conductive substrate and one or more membrane layers that produce electrical current in response to the amount of glucose present in the patient's body. The construction of these layers affects the performance of the device, such as the output readings and sensitivity. Manufacturing processes for CGM sensors can affect the accuracy and repeatability of fabricating these layers.

### BRIEF DESCRIPTION OF THE DRAWINGS

FIG. 1 is a not-to-scale cross-sectional view of a working wire, in accordance with some aspects.

FIG. 2 is an isometric view of a wire-holding fixture and a container for a solution, in accordance with some aspects.

FIG. 3A shows a first side of a wire-holding fixture, in accordance with some aspects.

FIG. 3B shows a second side of a wire-holding fixture, in accordance with some aspects.

FIG. 4A illustrates a staging area positioned adjacent to an electropolymerization (EP) chamber used for the electropolymerizing process, in accordance with some aspects.

FIG. 4B depicts the EP chamber of FIG. 4A, in accordance with some aspects.

FIG. 5A shows a front view of an example rack in the staging area, in accordance with some aspects.

FIG. 5B is a side view of the rack with wire-holding fixtures, in accordance with some aspects.

FIG. 6A is a top view of EP stations in an EP chamber, in accordance with some aspects.

FIG. 6B is a partial, close-up view of FIG. 6A, in accordance with some aspects.

FIG. 7 is a perspective view of an example of a hotplate with a magnetic stir feature as known in the art.

FIG. 8A shows a top close-up view of an EP station of the plurality of EP stations, in accordance with some aspects.

FIGS. 8B and 8C show perspective and top views, respectively, of a wire-holding fixture inserted into an EP station of the plurality of EP stations, both in accordance with some aspects.

FIG. 9 is a perspective view of an electrical contact board, in accordance with some aspects.

FIG. 10 is a not-to-scale side view schematic of the system during electropolymerization, in accordance with some aspects.

FIG. 11 is a flowchart describing an example method of electropolymerizing a material on a working wire, in accordance with some aspects.

FIG. 12 is an example of a CV plot for electropolymerization, in accordance with some aspects.

FIG. 13A is a schematic of an example of an electropolymerization system in accordance with some aspects.

FIG. 13B shows a longitudinal cross-sectional view of FIG. 13A, in accordance with some aspects.

FIG. 13C is a flowchart of a method for electropolymerizing a material onto a plurality of working wires for biological sensors in a continuous manner, in accordance with some aspects.

FIG. 14 is a simplified schematic diagram showing an example computer used to implement the controller, in accordance with some aspects.

### SUMMARY

A system for electropolymerizing a material onto a plurality of working wires for biological sensors is disclosed. The system includes a wire-holding fixture configured to be received in an electropolymerization (EP) station of the set of EP stations. The wire-holding fixture comprises a plurality of terminals, and each terminal is electrically coupled to a respective working wire of a plurality of working wires. The plurality of working wires extend away from the wire-holding fixture. A set of EP stations is included, and each EP station is configured to receive the wire-holding fixture. A container is configured to hold an electropolymerization solution and positioned beneath the EP station such that the plurality of working wires is immersible in the electropolymerization solution. A board comprising a plurality of electrical contacts is included, and each electrical contact is configured to electrically engage a corresponding terminal of the plurality of terminals. A controller is configured to apply a voltage to the plurality of working wires through the respective terminals and electrical contacts to initiate electropolymerization of the material from the electropolymerization solution to form a polymer layer on each working wire of the plurality of working wires.

A method for electropolymerizing a material onto a working wire for a biological sensor is disclosed. The method includes inserting a wire-holding fixture into an electropolymerization (EP) station of a set of EP stations. The wire-holding fixture is configured to hold a plurality of working wires and has a plurality of terminals. Each terminal of the plurality of terminals is coupled to a respective working wire of the plurality of working wires, and the plurality of working wires extend away from the wire-holding fixture. A container having an electropolymerization solution is positioned beneath the EP station of the set of EP stations. The plurality of working wires is immersed into the electropolymerization solution. A board having a plurality of electrical contacts is positioned in contact with the wire-holding fixture. Each electrical contact of the plurality of electrical contacts is configured to electrically engage with each terminal of the plurality of terminals. A controller applies a voltage to the plurality of working wires through the respective terminals and electrical contacts to initiate electropolymerization of the material from the electropolymerization solution forming a polymer layer on each working wire of the plurality of working wires.

### DETAILED DESCRIPTION

Systems and processes for manufacturing working wires for a continuous biological sensor are described herein, that enable manufacturing scalability and improve accuracy and efficiency compared to known art. The continuous biological sensor may be, for example, a continuous glucose monitor, in which the working wire includes an enzyme layer to detect the level of glucose in a patient's blood. In other aspects, the biological sensor may be a metabolic sensor for measuring other metabolic characteristics such as lactate, ketone or fatty acids. In some examples, the sensor uses a working wire (i.e., an electrode for the sensor) that has a core and several concentrically formed membrane layers on the core.

The systems and processes described herein enable electropolymerization of membrane layers of a sensor for a biological sensor, such as a glucose sensor for a continuous glucose monitoring device or a lactate sensor for a continuous lactate monitoring device, in an efficient and accurate manner, using careful control and feedback and with automated features. The membrane layers may be for an interference layer of a glucose sensor, for example, which may be formed by electropolymerization. The systems and processes uniquely provide mass production of biological sensors in a repeatable manner, and furthermore provide tracking of manufacturing parameters for each individual wire that is manufactured. This traceability is advantageous not only for keeping manufacturing records, but also enables precise calibration of each individual sensor by knowing the process conditions that were used for fabricating each particular sensor. As an example, the electropolymerization conditions for an individual sensor wire can be used to control the sensing layer thicknesses and consequently the sensitivity of the final sensor. This ability to predict the sensor's behavior over time can eliminate or reduce the need for local calibration by patients through finger stick monitoring, as is done with conventional CGM sensors.

The disclosed systems and methods provide a scalable and efficient approach to electropolymerizing material onto working wires for biological sensors. The present system enables batch processing of large numbers of wires simultaneously, such as 40, 60, or 80 wires per set of EP stations (also referred to as a bank), using a single shared electrical contact board. The electrical board is configured to engage and energize all wire-holding fixtures within a bank at once, significantly reducing system complexity and cost. Each bank may include multiple containers of electropolymerization solution, with each container capable of serving more than one wire-holding fixture, allowing parallel electropolymerization of dozens of working wires. The system is further configured to generate and monitor cyclic voltammetry (CV) curves in real time for each cycle, enabling evaluation of the electropolymerization process based on key features of the CV curve, such as peak voltage and area under the curve. This real-time monitoring facilitates verification of the electropolymerization process without requiring post-process inspection. It also enables identification of faults, such as when a fixture fails to make electrical contact, by detecting deviations from the expected voltage behavior. The system's ability to perform high-throughput, data-informed electropolymerization with minimal hardware overhead and enhanced process control represents a significant advancement in the scalable manufacturing of electropolymerization in biological sensors.

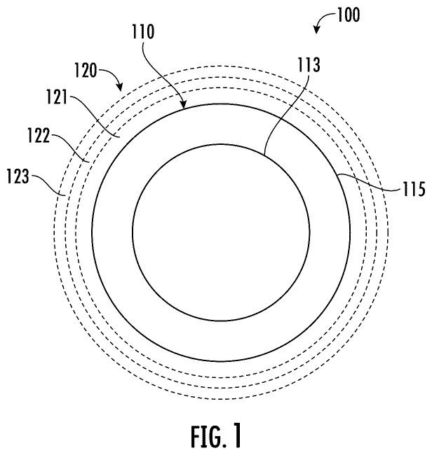

FIG. 1 is a not-to-scale cross-sectional view of a working wire, in accordance with some aspects. In this example, a working wire 100 is an elongated wire having a circular cross-section. It will be understood that other cross-sections may be used, such as square, rectangular, triangular, or other geometric shapes. Furthermore, the working wire 100 may take other forms, such as a plate or ribbon. The working wire 100 is used as a working electrode of a continuous biological sensor, such as a working electrode of a continuous glucose monitor.

In the illustrated example, the working wire 100 has a substrate 110 onto which biological membranes 120 may be disposed. In one example as illustrated, the biological membranes 120 include an interference membrane 121 (which may also be referred to as an interference layer) on the substrate 110, an enzyme membrane 122 (i.e., an enzyme layer) on the interference membrane 121, and a glucose limiting membrane 123 (i.e., a glucose limiting layer) on the enzyme membrane 122. In some aspects, a protective or outer coating may be optionally applied over the glucose limiting membrane 123. In cases where the working wire 100 is for a lactate sensor, glucose limiting membrane 123 may be instead configured as a lactate limiting layer. Although the working wire 100 is illustrated as having three biological membranes 120, it will be understood that the biological membranes 120 may be more or fewer in number.

The substrate 110 may be comprised of a core 113 with an outer layer 115. In the example of FIG. 1, the core 113 is an elongated wire that is dense, ductile, very hard, easily fabricated, highly conductive of heat and electricity, and may also be resistant to corrosion. Example materials for core 113 include tantalum (Ta), carbon (C), or cobalt-chromium (Co—Cr) alloys. The core 113 may have the outer layer 115, such as of platinum, deposited or applied using an electroplating process. It will be understood that other processes may be used for applying the outer layer 115 to the core 113. For a glucose monitor, the platinum outer layer facilitates a reaction where hydrogen peroxide reacts to produce water and hydrogen ions, and two electrons are generated. The electrons are drawn into the platinum by a bias voltage placed across the platinum wire and a reference electrode. In this way, the magnitude of the electrical current flowing in the platinum is intended to be related to the number of hydrogen peroxide reactions, which in turn is proportional to the number of oxidized glucose molecules. A measurement of the electrical current on the platinum wire can thereby be associated with a particular level of glucose in the patient's blood or interstitial fluid (ISF) (a biological fluid in the patient's body that contains diverse biomarkers and analytes and is similar to blood composition).

The core 113, outer layer 115, interference membrane 121, and enzyme membrane 122 form key aspects of the working wire 100. Other layers and/or membranes may be added depending upon the biological substance being tested for, and application-specific requirements. As described with reference to the working wire 100 of a metabolic sensor, glucose oxidase (GOx) may be used as the enzyme for detecting glucose levels. It will be understood that other enzymes, such as lactate oxidase or lactate dehydrogenase for lactate detection, may be used in metabolic sensors targeting other analytes. In some cases, the core 113 may have an inner core portion (not shown). For example, if the substrate (core 113) is made from tantalum (Ti), an inner core of titanium or titanium alloy may be included to provide additional strength and straightness for the working wire 100.

One or more membranes (i.e., layers) may be provided over the enzyme membrane 122. For example, the glucose limiting membrane 123 may be layered on top of the enzyme membrane 122. This glucose limiting membrane 123 may limit the number of glucose molecules that can pass through the glucose limiting membrane 123 and into the enzyme membrane 122. The glucose limiting membrane 123 can be configured as described in U.S. Pat. No. 11,576,595, entitled “Enhanced Sensor for a Continuous Biological Monitor,” which is owned by the assignee of the present disclosure and is incorporated herein by reference as if set forth in its entirety. In some cases, the addition of the glucose limiting membrane 123 has been shown to enable better performance of the overall working wire 100.

The interference membrane 121 is applied over the outer layer 115 of the substrate 110. The interference membrane 121 may be disposed between the enzyme membrane 122 and the outer layer 115. This interference membrane 121 is constructed to fully wrap the outer layer 115, thereby protecting the outer layer 115 from, or mitigating, oxidation effects. The interference membrane 121 is also constructed to substantially restrict the passage of larger molecules, such as acetaminophen, to reduce contaminants that can reach the platinum of the outer layer 115 and skew results. Further, the interference membrane 121 may pass a controlled level of hydrogen peroxide (H₂O₂) from the enzyme membrane 122 to the platinum outer layer 115. Compositions for the interference membrane 121 and the enzyme membrane 122 may be as described in U.S. patent application Ser. No. 19/047,285, entitled “Continuous Biological Sensor with Enzyme Immobilization,” and U.S. patent application Ser. No. 17/449,380, entitled “In-Vivo Glucose Specific Sensor,” which are owned by the assignee of the present disclosure and incorporated herein by reference as if set forth in their entirety.

The interference membrane 121 may be electrodeposited onto the electrical conducting wire (i.e., substrate 110) in a very consistent and conformal way, thus reducing manufacturing costs as well as providing a more controllable and repeatable layer formation. The interference membrane 121 is formulated to be nonconducting of electrons but will pass negative ions at a preselected rate. Further, the interference membrane 121 may be formulated to be permselective (i.e., semipermeable) for particular molecules. In one example, the interference membrane 121 is formulated and deposited in a way to restrict the passage of larger molecules, which may act as contaminants to degrade the conducting layer (i.e., the substrate 110), or that may interfere with the electrical detection and transmission processes.

In one example, a particularly effective interference membrane 121 may be constructed using, for example, pyrrole and phenylenediamine (PDA) deposited onto the platinum wire (e.g., working wire 100) using an electrodeposition process, at a thickness that can be precisely controlled to enable a predictable level of hydrogen peroxide to pass through the interference membrane 121 to the platinum electrode.

The interference membrane compound is formulated to be non-electrically conducting, ion passing, and permselective. The interference membrane compound is particularly formulated to be electrodeposited in a thin and uniform layer, and that has a thickness that is self-limiting due to the nature of electrically driven cross-linking. In this way, the interference membrane compound may be applied in a way that provides a well-controlled regulation of hydrogen peroxide molecule passage using a simple and cost-effective manufacturing processes. Further, the passage of the hydrogen peroxide can occur over a much larger surface area as compared to prior art working wires.

The interference membrane compound may be electrodeposited onto the conductive substrate 110, in which the electropolymerization deposits the interference membrane compound in a thin and uniform layer. The electrodeposition process facilitates a chemical cross-linking of materials in the interference membrane compound such as polymers, as the polymer is deposited. It will be understood that other processes may be used to apply the interference membrane compound to create the interference membrane 121 to the conductive substrate 110.

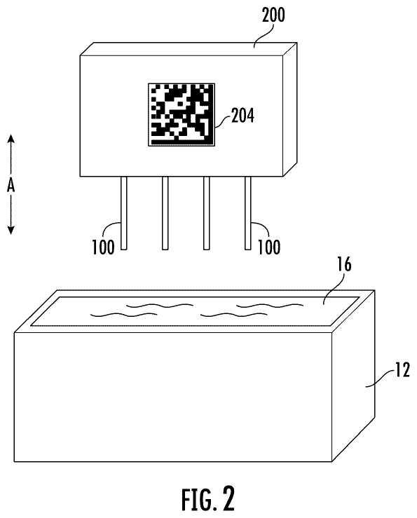

FIG. 2 is an isometric view of a wire-holding fixture and a container for holding a solution, in accordance with some aspects. A plurality of working wires 100 are mounted into a wire-holding fixture 200 where the working wires 100 may be uncoated or may have some but not all the membrane layers coated onto it. For example, working wires 100 may consist of only the substrate 110 (of FIG. 1), or may be in the process of dipping layers of the interference membrane 121, and enzyme membrane 122, or glucose limiting membrane 123 onto the substrate 110. The wire-holding fixture 200, depicted as a block in this illustration, functions as a holder for transporting the working wires 100 through a coating process, such as electropolymerization, during manufacturing as described in this disclosure.

The container 12 holds a coating solution 16, such as an electropolymerization solution comprising a polymer or polymer mixture used for electropolymerization, as described in this disclosure. Alternatively, for lactate monitors (i.e., lactate sensors) the coating solutions in the container 12 may be a lactate solution. The working wires 100, mounted on the wire-holding fixture 200, are at least partially submerged, immersed, or otherwise introduced into the coating solution 16 to form a desired membrane layer on the surface of the wires through a coating process such as electropolymerization. The relative movement between the wire-holding fixture 200 and the container 12 is shown by arrow A. In some examples, the wire-holding fixture 200 may be moved toward the coating solution 16, and in other examples, the container 12 may be moved toward the wire-holding fixture 200. The electropolymerization process may be utilized to form the interference membrane 121 or another functional membrane of the biological sensor, such as a membrane in a glucose sensor used in a continuous glucose monitoring system or a membrane in a lactate sensor. Each membrane may require multiple electropolymerization cycles, with several coating iterations applied to build up a desired thickness of the membrane. The wire-holding fixture 200 may include an identifier code 204 to facilitate tracking throughout various stages of the manufacturing process. The identifier code 204 may comprise a scannable code, such as a barcode or quick response (QR) code, that can be optically read by a scanner, camera, reader, optical device or imaging device (e.g., optical reader). In some examples, the identifier code 204 may alternatively or additionally include other types of identifying labels, such as a radiofrequency identification (RFID) tag or a near-field communication (NFC) element, to enable automated, contactless identification and data logging during manufacturing operations.

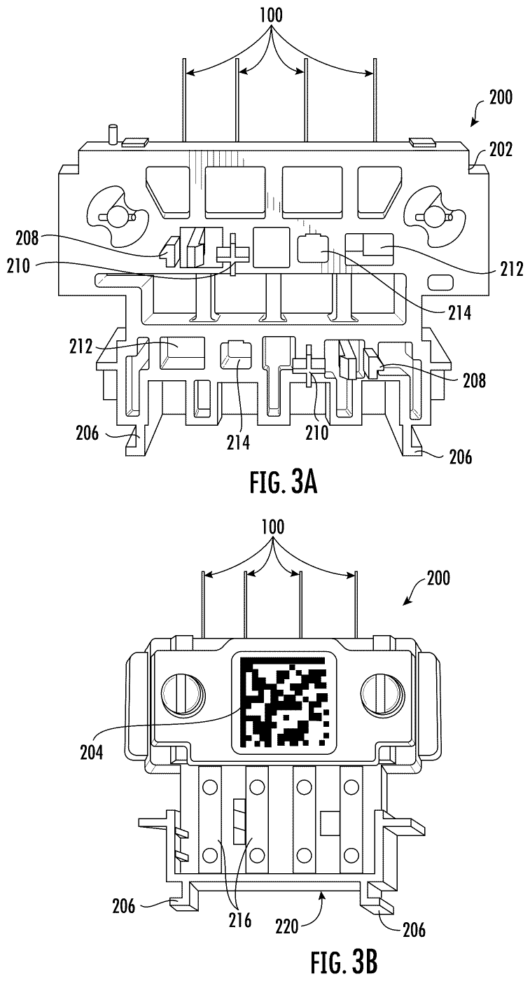

FIG. 3A shows a first of a wire-holding fixture, and FIG. 3B shows a second side of a wire-holding fixture, both in accordance with some aspects. In FIG. 3A, four working wires 100 are shown mounted into a body 202 of the wire-holding fixture 200. In other examples, the wire-holding fixture 200 may be configured to hold more or fewer working wires during manufacturing, such as 1 to 10, or 2 to 8, or 2 to 6, or other appropriate number or range. The wire-holding fixture 200 is a holder for transporting the working wires through various steps during manufacturing (e.g., electropolymerization coating, dipping, calibration, etc.). The wire-holding fixture 200 may also be referred to as a carrier or tray. The working wires may be secured into the wire-holding fixture 200 by, for example, clamps, spring-loaded clips, set screws, adhesive fasteners, or other mechanisms.

The working wires 100 may be mounted in a single row on the wire-holding fixture 200, spaced apart and extending from an edge of the fixture to allow each wire to be individually accessed and measured from various angles. In other configurations, the working wires may be arranged in multiple rows, either aligned or staggered relative to one another, provided that sufficient spacing is maintained between adjacent wires to permit individual measurement, testing, or calibration of each working wire without interference.

The wire-holding fixture 200 has L-shaped feet 206 to enable it to stand, be fixed in place, or be hung upside down as needed. Snapping tabs 208 are also included, extending outward from the face of the wire-holding fixture 200, to enable two wire-holding fixtures 200 to be snapped together (e.g., in pairs, back-to-back) and transported together during manufacturing and/or testing/calibration. In this illustration, two sets of snapping tabs 208 and alignment posts 210 are positioned diagonally from each other on the wire-holding fixture 200. When two wire-holding fixtures 200 are placed with the same sides facing each other, a snapping tab 208 on one wire-holding fixture 200 fits into a tab hole 212 on the other wire-holding fixture 200 that it is being attached to (i.e., a mating fixture), and the alignment post 210 fits into an alignment hole 214 of the mating fixture.

The diagonal positioning of the alignment posts 210 on the wire-holding fixtures 200 helps ensure that the wire-holding fixtures 200 are properly aligned in all axes, and the snapping tabs 208 lock the wire-holding fixtures 200 together. Being able to thus “gang” or attach wire-holding fixtures 200 together allows more than one wire-holding fixture 200 to be moved together at a time (e.g., by a robot), which increases manufacturing speed. Various features of the wire-holding fixture 200 such as wings, indentations, grooves, and the like, may be used as gripping features for a robot to grab. The features may be configured to provide proper alignment in a robot (e.g., in x-y-z linear and rotational directions) and when inserted into a manufacturing process (e.g., dipping, coating, or electrical testing/calibration), since misalignment can affect the uniformity and/or concentricity of the coatings on the working wires 100 or electrical contact of the wire-holding fixture 200 with a test/calibration apparatus.

In FIG. 3B, the opposite face of the wire-holding fixture 200 as shown in FIG. 3A is illustrated. The identifier code 204, which may include a QR code, barcode, RFID tag, or other machine-readable identifier, is included for tracking the wire-holding fixture 200 during manufacturing and testing/calibration as described above. In this example, the identifier code 204 is a QR code sticker that is affixed to a plate (e.g., metal) that is attached to the body 202 (e.g., plastic). Additionally, a plurality of terminals 216 are also shown attached to the top of the body 202. One terminal 216 is present for and electrically connected to a corresponding working wire 100 in the wire-holding fixture 200. The plurality of terminals 216 provide an electrical connection between the working wire 100 and a terminal point on a bottom side 220 of the wire-holding fixture 200 for various manufacturing process steps, such as electropolymerization and electrical testing (e.g., calibration). In other words, the plurality of terminals 216 provide an electrical connection between each working wire 100 and a corresponding contact region on a bottom side 220 of the wire-holding fixture 200, enabling external electrical interface during manufacturing process steps such as electropolymerization and electrical testing, including calibration. The plurality of terminals 216 are shown as metal strips; however, in other examples, the plurality of terminals 216 may comprise wires, electrically conductive coatings, conductive traces, metal pads, spring contacts, or other suitable electrical conduits configured to establish electrical connection between the working wires and external components.

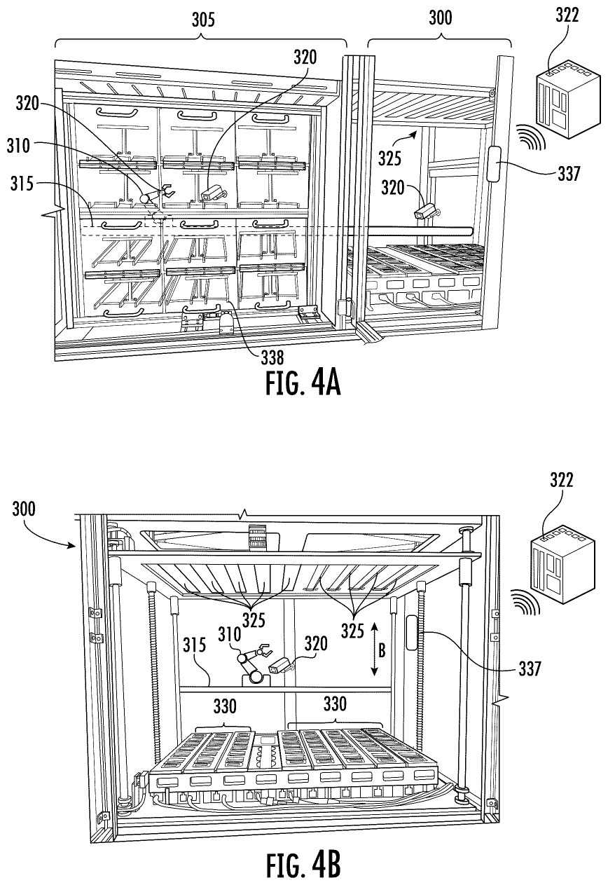

FIG. 4A illustrates a staging area positioned adjacent to an electropolymerization (EP) chamber used for the electropolymerizing process, and FIG. 4B depicts the EP chamber itself of FIG. 4A, both in accordance with some aspects. A staging area 305 is configured to store wire-holding fixtures 200 containing working wires 100 on racks 338 before and after the electropolymerization process. A robotic mechanism, or robot 310, such as a 3-axis robot or 6-axis robot 310 with a robotic manipulation arm, may be used to move the wire-holding fixtures 200 between the staging area 305 and the EP chamber 300. The robot 310 may be mounted on and configured to move along a rail 315, enabling it to retrieve a wire-holding fixture 200 from the staging area 305 and transport it along the rail to the EP chamber 300, where it loads the wire-holding fixture 200 into an EP station of a plurality of EP stations 330.

One or more optical readers 320 may be positioned at various locations within the staging area 305 and the EP chamber 300 to monitor and track the position of wire-holding fixtures 200 during the manufacturing process. Each optical reader 320 is configured to read an identifier code 204 on a corresponding wire-holding fixture 200. The optical reader 320 is in communication with a controller 322. As used herein, a controller 322 may refer to any suitable processing unit, computer, microcontroller, or processor configured to execute instructions, manage data, and control one or more components of the system. The controller 322 is configured to store manufacturing data associated with each working wire 100 based on the identifier code 204. By reading the identifier codes 204, the optical reader 320 enables tracking of each wire-holding fixture 200, allowing the system to record detailed manufacturing information, such as which wire-holding fixtures 200 are undergoing electropolymerization and the specific process parameters applied to each wire-holding fixture 200 and its associated working wires 100 during each stage of operation.

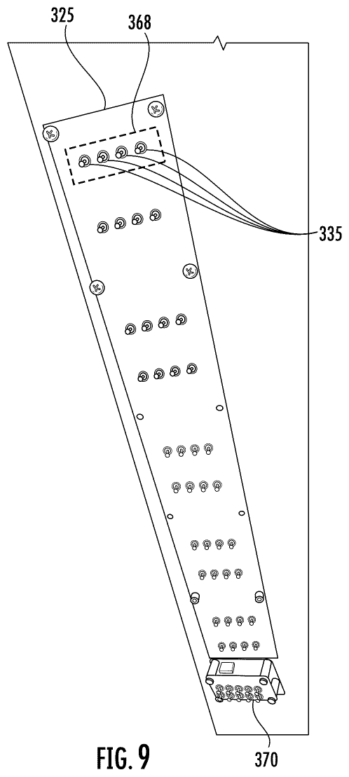

An electrical contact board 325 is positioned above a plurality of electropolymerization (EP) stations 330 and is configured to move relative to the set of EP stations, as indicated by arrow B. In the illustrated example, the electrical contact board 325 moves vertically and is shown in a raised position in FIGS. 4A and 4B to allow wire-holding fixtures 200 to be loaded into the EP stations 330. During electropolymerization, the electrical contact board 325 moves downward toward the EP stations 330 to establish electrical contact. The electrical contact board 325 includes a plurality of electrical contacts 335 (shown in FIG. 9), each configured to engage a corresponding wire-holding fixture 200 positioned in the EP stations 330. A mechanism 337 is configured to move the electrical contact board 325 into and out of contact the plurality of terminals 216 of the wire-holding fixture 200. The mechanism 337 for movement of the electrical contact board 325 may be an actuator, such as a linear screw, pneumatic piston, hydraulic piston, or any other suitable motion mechanism.

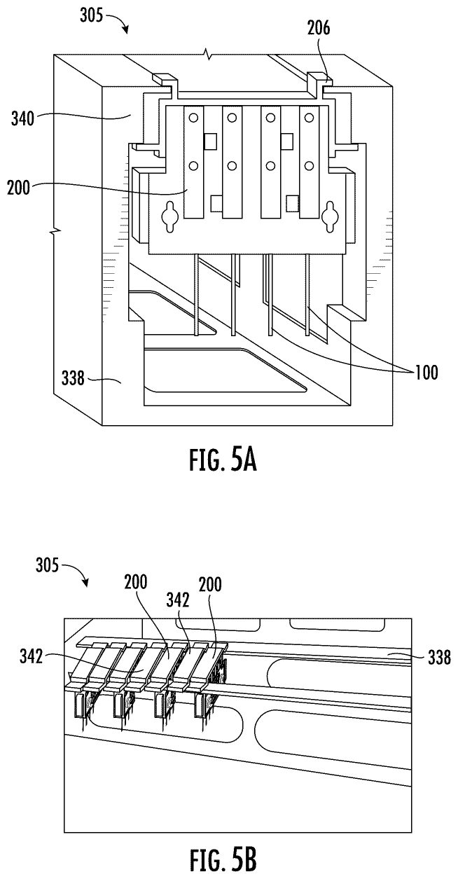

FIG. 5A shows a front view of an example rack in the staging area, and FIG. 5B is a side view of the rack with wire-holding fixtures, both in accordance with some aspects. The rack 338 in the staging area 305 holds a wire-holding fixture 200 for working wires 100. The wire-holding fixture 200 is oriented upside down compared to FIGS. 3A and 3B, such that the working wires 100 extend downward from the bottom edge of the wire-holding fixture 200 in FIG. 5A. The corresponding terminals 216 on the wire-holding fixture 200 are also visible. The feet 206 of the wire-holding fixture 200 may rest on upper rails 340 of the rack 338, allowing multiple wire-holding fixtures 200 to be slid into place and securely held within the rack 338. During manufacturing, multiple wire-holding fixtures 200 may be arranged on the rack 338. Spacers 342 may be inserted between wire-holding fixtures 200 in this illustration. The spacers 342 are shaped similarly to the wire-holding fixtures 200 but are not configured to hold any working wires. In other words, the spacers 342 are dummy fixtures that serve to separate the working wires 100 of adjacent wire-holding fixtures 200 from each other, to help prevent damage to the working wires 100. The robot 310 may retrieve each wire-holding fixture 200 from the rack 338 and transfer them through the electropolymerization process.

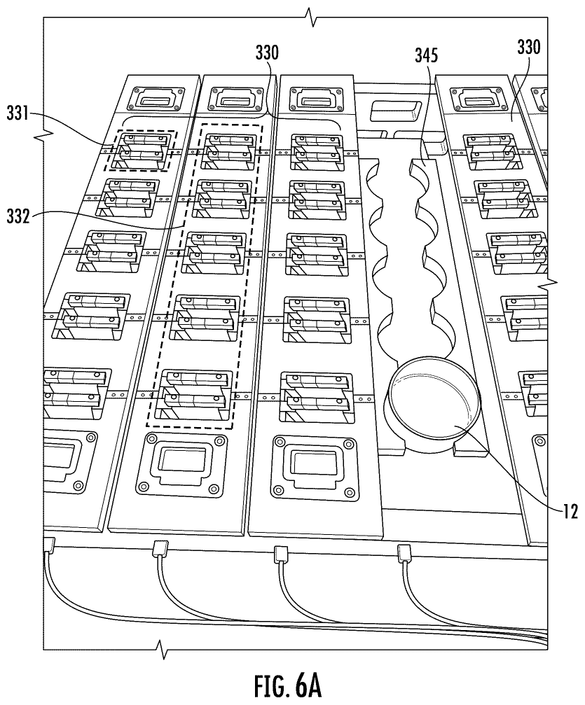

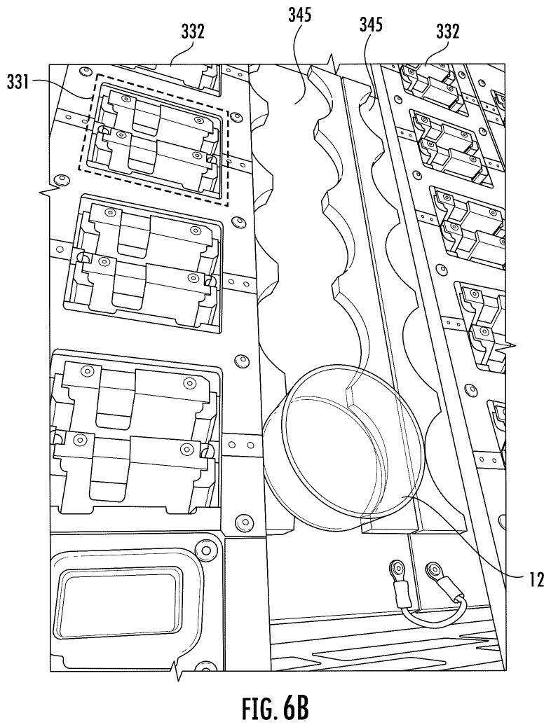

FIG. 6A is a top view of EP stations in an EP chamber, and FIG. 6B is a partial, close-up view of FIG. 6A, both in accordance with some aspects. Each EP station 331 of the plurality of EP stations 330 in the EP chamber 300 is configured to receive wire-holding fixtures 200. In the illustrated example, each EP station 331 accommodates two wire-holding fixtures 200. A set of EP stations 332, also referred to as a bank, comprises a row of multiple individual EP stations 331, as indicated by broken lines in the figure. In some examples, the EP stations in the set of EP stations 332 are arranged in a linear array and the electrical contact board 325 has a corresponding linear array of electrical contacts 335. In this example, each set includes five EP stations, although in other examples the number of EP stations per set may vary. Additionally, one set of EP stations 332 is omitted from the illustration to expose a plate 345 positioned beneath each set of EP stations 332.

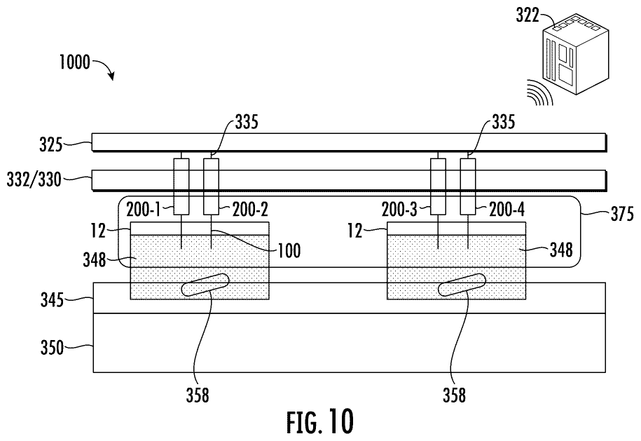

The plate 345 is configured to support a plurality of containers 12 and includes shaped recesses sized and contoured to receive the containers. The shape of each recess may correspond to the shape of the respective container 12 to ensure stable placement. The containers 12 may include crystallization dishes, beakers, bowls, dishes, or tubs, and are used to hold a coating solution, such as electropolymerization solution 348 (FIG. 10), for coating the working wires 100. It is understood that the composition of the electropolymerization solution may vary depending on the target analyte. For example, in the case of a lactate sensor for a continuous lactate monitoring device, the solution may be formulated to incorporate monomers and components specific to lactate detection. In the illustrated figure, only one container 12 is shown to provide a clear view of the underlying plate 345. In some examples, each container 12 is configured to hold two wire-holding fixtures 200, enabling five containers to be placed on plate 345 to accommodate a total of ten wire-holding fixtures 200 in a single row of EP stations 331 (i.e., a set of EP stations 332). Similar plates 345 are positioned beneath each set of EP stations 332, as illustrated in FIG. 6B.

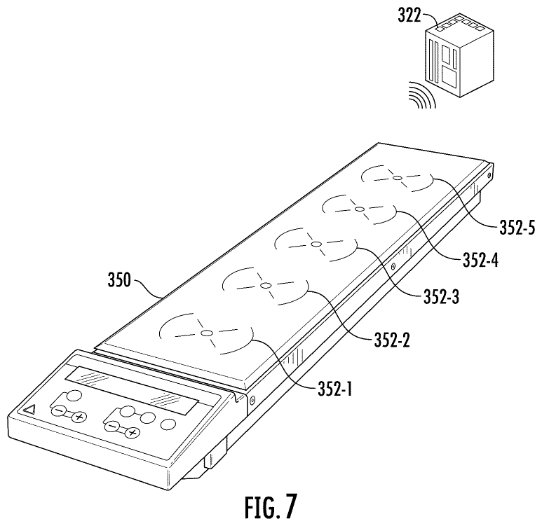

FIG. 7 is a perspective view of an example of a hotplate with a magnetic stir feature as known in the art. In some examples, a hotplate 350 may be a combined hotplate and a magnetic stirrer, a magnetic stirrer hotplate, a hotplate without stirring functionality, or a stir plate without heating capability. The hotplate 350 is positioned below or beneath the plate 345 (shown in FIGS. 6A and 6B), where the plate 345 holds the containers 12 filled with electropolymerization solution 348. In some examples, the plate 345 is thermally conductive. The hotplate 350 is in communication with the controller 322, and the controller 322 is configured to control the temperature of the electropolymerization solution 348 via the hotplate 350. For example, the hotplate 350 may be used to adjust the temperature of the electropolymerization solution 348 to a desired temperature. In some examples, the controller 322 is configured to control a temperature of the electropolymerization solution 348 via the hotplate 350 positioned beneath the plate 345 and in communication with the controller 322.

The hotplate 350 may include, or be used in conjunction with, stirring functionality to operate with a stirrer positioned in the container 12 and hence, in the electropolymerization solution 348. The stirrer may be a magnetic stirring bar 358 (shown in FIG. 10) and in communication with the controller 322. The controller 322 may be configured to control a stirring rate of the magnetic stirring bar 358 (e.g., stirrer). Stirring promotes a uniform distribution of the materials within the electropolymerization solution 348 and helps prevent the formation of concentration gradients through the depth of the solution as material is deposited near the surface onto the working wires 100.

The example hotplate 350 shown in FIG. 7 is a RT 5 model from IKA Works, Inc., which features five positions (352-1, 352-2, 352-3, 352-4, 352-5) for holding containers 12, corresponding to the five container positions on the plate 345 shown in FIGS. 6A and 6B. The hotplate 350 may be made of a thermally conductive material, such as aluminum, to help ensure uniform temperature across all five containers. Temperature control is critical during electropolymerization, as reaction rates are temperature dependent. For thin-film coatings used in biological sensors (e.g., layer thicknesses on the order of microns) uniform temperature is essential for achieving consistent layer thickness across all wire-holding fixtures 200 in a set of EP stations 332. In addition to temperature control, the magnetic stirring feature of the hotplate 350 improves deposition uniformity. As the polymer material in the electropolymerization solution 348, such as PDA, is consumed near the surface during the electropolymerization process, a concentration gradient may form. Stirring with the magnetic stirring bar 358 helps to continuously mix the electropolymerization solution 348, reducing this gradient and promoting more consistent coating of the working wires 100.

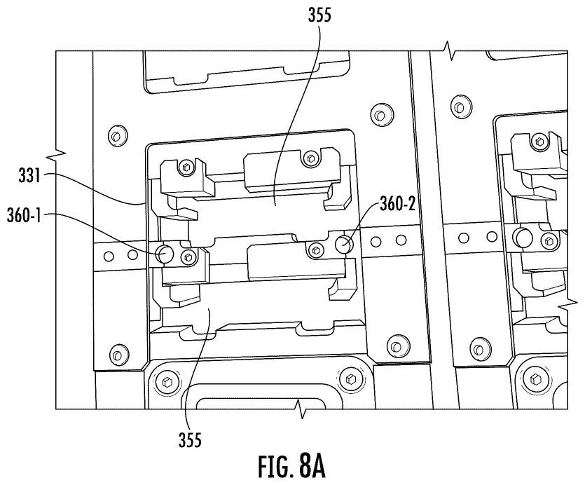

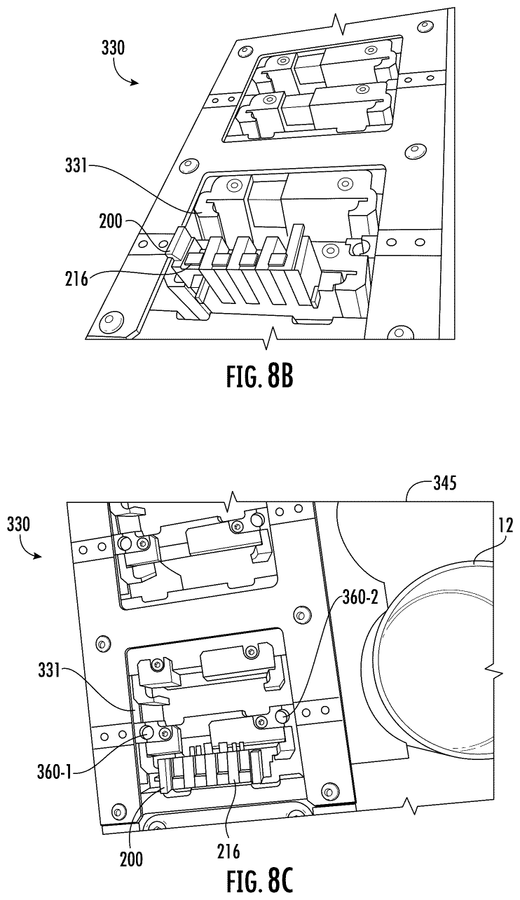

FIG. 8A shows a top close-up view of an EP station of the plurality of EP stations, in accordance with some aspects. Each EP station 331 of the plurality of EP stations 330 has at least one slot 355 in which a wire-holding fixture 200 for working wires 100 will be inserted. A set or pair of electrodes, 360-1 and 360-2 respectively, are included on the EP station 331. Electrode 360-1 may function as a reference electrode, and electrode 360-2 may function as a counter electrode, both configured to provide electrical voltage to the wire-holding fixtures 200 during the electropolymerization process. Each EP station 331 has a set of electrodes, 360-1 and 360-2, configured to apply voltage across to the wire-holding fixture 200 positioned within the EP station 331. In this example, electrode 360-1 and electrode 360-2 apply voltage to the wire-holding fixtures 200 positioned adjacent to them. In some cases, each EP station 331 of the plurality of EP stations 330 includes two slots 355, with each slot 355 configured to receive a wire-holding fixture 200 containing multiple working wires 100. The electrodes 360-1 and 360-2 are configured to apply voltage to both wire-holding fixtures 200 positioned in the EP station, thereby enabling electropolymerization on all of the working wires 100 held within the wire-holding fixtures 200. In some examples, each working wire may be electrically biased individually, all of the wires within a single wire-holding fixture 200 may be biased simultaneously as a group, or all of the wires across both wire-holding fixtures 200 in the EP station 331 may be biased together.

FIGS. 8B and 8C show perspective and top views, respectively, of a wire-holding fixture inserted into an EP station of the plurality of EP stations, both in accordance with some aspects. The terminals 216, previously described in FIG. 3B, are visible in these views. These terminals 216 function as electrical terminals for delivering voltage to each of the working wires 100 mounted in the wire-holding fixture 200. Electrodes 360-1 and 360-2 act as electrical conduits that supply power to each EP station 331 of the plurality of EP stations 330, enabling electropolymerization across the associated working wires 100.

FIG. 9 is a perspective view of an electrical contact board, in accordance with some aspects. Referring to FIGS. 4A, 4B, and 9, a plurality of electrical contact boards 325 are positioned above the plurality of EP stations 330 within the EP chamber 300. Each electrical contact board 325 includes a plurality of electrical contacts 335 that are configured to engage the wire-holding fixtures 200. The plurality of electrical contacts 335 may be spring-loaded pogo pins, allowing for quick and reliable connection and disconnection of the wire-holding fixtures 200. In some examples, each set of EP stations 332 (see FIG. 6A) is paired with a corresponding electrical contact board 325. Each working wire 100 in a wire-holding fixture 200 aligns with a dedicated electrical contact of the plurality of electrical contacts 335. In the illustrated configuration, each set of electrical contacts 368 of the plurality of electrical contacts 335 includes four pogo pins (e.g., electrical contacts 335), corresponding to the four working wires 100 to be coated in each wire-holding fixture 200. The system includes five groups of two sets of electrical contacts, corresponding to the ten EP stations in each set of EP stations 332. A junction board 370, positioned at the end of each electrical contact board 325, is configured to supply power to the electrical contacts 335.

FIG. 10 is a not-to-scale side view schematic of a system during electropolymerization, in accordance with some aspects. A system 1000 includes at least one EP station 331 or a set of EP stations 332 of the plurality of EP stations 330. A portion of a set of EP stations 332 (e.g., two EP stations) is shown with four wire-holding fixtures 200 (200-1, 200-2, 200-3 and 200-4) for two containers 12 of electropolymerization solution 348 (e.g., polymer solution) are illustrated. During electropolymerization, the electrical contact board 325 is moved downward or toward the wire-holding fixtures 200 (from its upward position in FIG. 4A) so that the electrical contacts 335 are in connection with the terminals 216 (e.g., terminals) on the wire-holding fixtures 200. The wire-holding fixtures 200 are seated in the slots 355 of each EP station 331 of a set of EP stations 332 of the plurality of EP stations 330 such that the working wires 100 that are to be coated extend from the wire-holding fixtures 200 and toward the electropolymerization solution 348 in the containers 12. The working wires 100 are immersed in the electropolymerization solution 348 of the containers 12 to be electropolymerized. The magnetic stirring bar 358 in each container 12 helps to maintain a uniform distribution of monomers in the electropolymerization solution 348 as the electropolymerization cycles are repeated over time. The containers 12 are in contact with the plate 345, which, in some examples, is positioned on the hotplate 350 as described herein. The set of EP stations 332, electrical contact board 325, wire-holding fixtures 200, magnetic stirring bar 358 and hotplate 350 are in communication with the controller 322.

In some examples, a curtain or blanket of inert gas 375 (e.g., nitrogen) is placed over the containers 12 and may be utilized to help improve the quality of the electropolymerization. In one example, the inert gas 375 can eliminate the reaction of oxygen from ambient air with the monomers in the electropolymerization solution 348, such as phenylenediamine (PDA). The inert gas 375 may be supplied from an external gas tank, where the gas fills the space between the plate 345 and the plurality of EP stations 330.

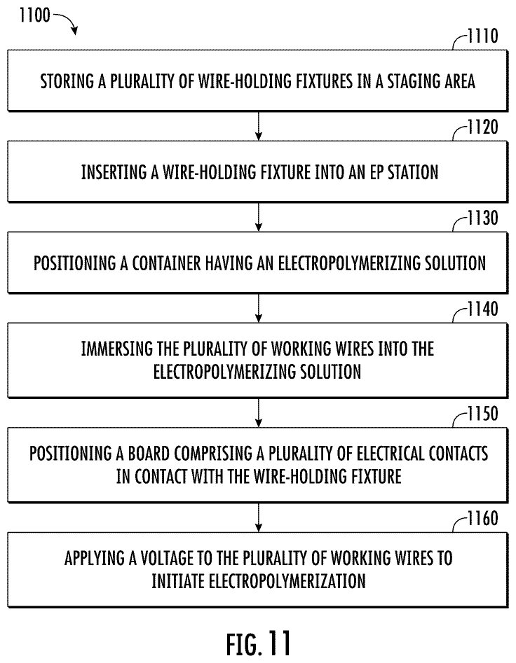

FIG. 11 is a flowchart describing an example method of electropolymerizing a material on a working wire, in accordance with some aspects. The particular steps, order of steps, and combination of steps are shown for illustrative and explanatory purposes only. Other aspects can implement different particular steps, orders of steps, and combinations of steps to achieve similar functions or results. A method 1100 for electropolymerizing a material onto a plurality of working wires for biological sensors uses the systems described herein. At block 1110, wire-holding fixtures 200 that have working wires 100 loaded into them are stored in a staging area 305. In some examples, the wire-holding fixture 200 is configured to hold four or more working wires 100. A robot 310 transports the wire-holding fixture 200 between the staging area 305 and the EP station 331 of the set of EP stations 332.

At block 1120, the wire-holding fixture 200 is inserted into the slot 355 of the EP station 331. In some examples, this may be accomplished manually or by a robot 310 in communication with the controller 322. The controller 322 controls the range of motion of the robot 310. The wire-holding fixture 200 is configured to hold a plurality of working wires 100 and comprises a plurality of terminals 216. Each terminal 216 of the plurality of terminals 216 is coupled to a respective working wire 100 of the plurality of working wires 100. The plurality of working wires 100 extends away from the wire-holding fixture 200.

An optical reader 320 reads each wire-holding fixture's identifier code 204 and sends information to the controller 322 about which EP station 331 each individual wire-holding fixture 200 was placed into. This and other manufacturing data associated with the working wire 100 associated with the identifier code 204 may be stored by the controller 322. The optical reader 320 is positioned within the system 1000, for example, on a robotic arm, on a wall of the EP chamber 300 or staging area 305, or in proximity to the EP stations. The optical reader 320 is configured to read the identifier code 204 located on each wire-holding fixture 200.

At block 1130, a container 12 having an electropolymerization solution 348 is positioned beneath the EP station 331 of the set of EP stations 332. In some aspects, the container 12 is placed on a plate 345 configured to hold a plurality of containers 12. In some aspects, the plate 345 is heated using a hotplate 350 positioned beneath the plate 345. The hotplate 350 may comprise a thermally conductive material and may be in communication with the controller 322. The controller 322 may be configured to control the temperature of the electropolymerization solution 348 via the hotplate 350. In some aspects, the electropolymerization solution 348 may be stirred using a magnetic stirring bar 358, and the stirring rate may be controlled by the controller 322.

At block 1140, the plurality of working wires 100 is immersed into the electropolymerization solution. At block 1150, a board 325 comprising a plurality of electrical contacts 335 is positioned in contact with the wire-holding fixture 200. Each electrical contact 335 of the plurality of electrical contacts 335 is configured to electrically engage with each terminal 216 of the plurality of terminals 216.

At block 1160, an electropolymerization cycle is performed by applying a voltage to the wires, which causes materials from the electropolymerization solution 348 in the container 12 to polymerize onto the working wires 100. For example, the controller 322 applies a voltage to the plurality of working wires 100 through the respective terminals 216 and electrical contacts 335 to initiate electropolymerization of the material from the electropolymerization solution 348 forming a polymer layer on each working wire 100 of the plurality of working wires 100. In some aspects, the electropolymerization solution 348 may be a mixture of PDA and pyrrole. The applying of the voltage to the plurality of working wires 100 to initiate electropolymerization is repeated until a target polymer thickness is achieved on each working wire 100. Once this is achieved, the wire-holding fixtures 200 are unloaded from the EP station 331 by the robot 310 and transported into the staging area 305 or moved to the next manufacturing process station. For example, the current is monitored while the applying of the voltage. The applying of the voltage is repeated (e.g., cycles) until the current decreases to a minimal or near-zero level, indicating that the reaction has reached a self-limiting state. Additional cycles may be applied at this stage to ensure uniformity and full completion of the polymer layer.

The electropolymerization parameters comprise at least one of the voltage, a current, a cycle time, a cycle count, or a voltammetry profile. These are collected for each individual working wire 100 through the electrical contacts 335 (e.g., pogo pins) and recorded for each working wire 100 via the controller 322. The collected data may include the electropolymerization reaction (i.e., cyclic voltammetry curve) for each cycle. These parameters may be associated with the identifier code 204.

In some examples, the interference membrane 121 is made of a compound that is self-limiting in thickness. The overall allowable thickness for the interference membrane 121 may be adjusted according to the ratio between the monomer(s) and a buffer, as well as the particular electrical characteristics used for the electropolymerization process. Also, the interference membrane 121 may be formulated for a particular permselective characteristic by adjusting the pH. It will also be understood that a cyclic voltammetry (CV) process may be used to electrodeposit the interference membrane compound. A CV process is generally defined by having (1) a scanning window that has a lower voltage limit and upper voltage limit, (2) a starting point and direction within that scanning window, (3) an elapsed time for each cycle, and (4) the number of cycles completed. It will be understood by one skilled in the art that these four factors can provide nearly infinite alternatives in the precise application of the interference membrane compound.

During electropolymerization, an electrical potential is applied across the working wires 100 and corresponding electrodes while the wires 100 are immersed in an electropolymerization solution 348 containing one or more monomers, such as pyrrole or phenylenediamine (PDA). The applied voltage initiates an electrochemical reaction at the surface of the working wire 100, resulting in the polymerization and deposition of the monomer onto the wire 100 to form a thin membrane layer.

The process may be performed using techniques such as cyclic voltammetry, where the applied voltage is swept through a defined range while the resulting current is measured to monitor the progression of the electropolymerization reaction. As the polymer layer forms and thickens, the current response associated with monomer oxidation or reduction decreases, indicating a decline in electrochemical activity at the wire surface. This behavior reflects the self-limiting nature of the electropolymerization process, where further monomer deposition is naturally inhibited by the growing polymer film.

When the measured current approaches zero or stabilizes at a minimal value, this indicates that the electropolymerization reaction has effectively completed or reached a self-limiting endpoint. In some implementations, between 15 and 30 voltage cycles may be applied, such as 20 cycles as in one example. Additional voltage cycles may be applied beyond this point to ensure uniform coverage and full completion of the polymerization process. Because the growth of the polymer film is governed by electrochemical activity and limited by material diffusion and charge transfer, this process enables precise control over film thickness, reproducibility, and membrane uniformity across multiple wires.

In one example, the electropolymerization process is performed using a voltage sweep ranging from approximately −0.4 volts to +0.8 volts. This voltage range is sufficient to initiate the electrochemical polymerization of the monomers. The resulting current response is monitored throughout the cycle to assess the progression of the reaction. As the polymer layer forms and the monomer is consumed, the current decreases, indicating reduced electrochemical activity and confirming the self-limiting nature of the process.

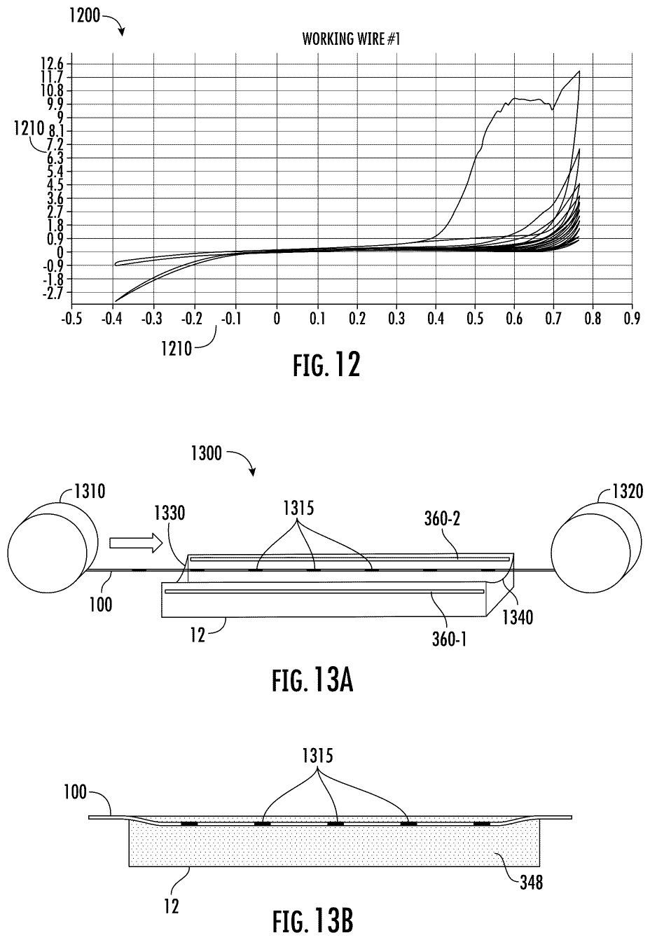

FIG. 12 is an example of a CV plot for electropolymerization, in accordance with some aspects. A CV plot 1200 shown is of PDA and pyrrole, where voltage 1210 is on the X-axis and the resulting current 1220 is on the Y-axis. The total number of CV cycles may be, for example, 5-50, such as 15-25. Each cycle gets lower because as the membrane layers accumulate, the coated wire becomes less conductive. Thus, the choice of polymer compound in this example results in the process being self-insulating (and therefore self-limiting). The CV data may be collected and stored for each working wire 100 by associating the data with the wire-holding fixture's identifier code 204 and the wire's location in each wire-holding fixture 200.

The CV data and other EP parameters may be used to help calibrate and/or predict a sensitivity of the finished sensor over time, when used in the field, thereby resulting in biological sensors (e.g., CGM sensors) with improved accuracy and performance compared to conventional sensors. Furthermore, the sensors do not need to be repeatedly calibrated by the patient because the performance of the present sensors (e.g., changes in sensitivity over time) can be predicted by using the tracked manufacturing data for each individual working wire. In contrast, conventional CGM sensors must be locally calibrated by each patient prior to use and then re-calibrated over time for a particular user, to account for variability in sensitivity of CGM sensors over time. Unfortunately, the local calibration processes require the patient to prick their finger and obtain a blood glucose reading using a standard strip monitor. Not only is local calibration inconvenient, time consuming, and prone to error, it can also be painful such that a patient may delay or avoid local calibration, thereby defeating any possible benefit from the CGM system.

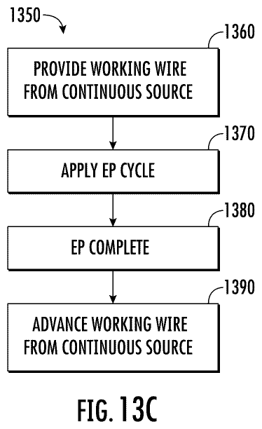

FIG. 13A is a schematic of an example of an electropolymerization system in accordance with some aspects. In this example, a system 1300 may process the working wires 100 as a continuous length of wire, such as wire fed from a spool, rather than as discrete or individual segments as illustrated in FIG. 3A. A continuous process may be advantageous by requiring less handling of, and therefore less potential damage to, pieces of wires that are cut to short lengths. In this example of system 1300, the working wire 100 is fed from a spool 1310 horizontally, along the length of an elongated container 12, such as a trough, tub, channel, or other longitudinal vessel configured to accommodate extended wire lengths during processing. The container 12 holds the electropolymerization solution 348, and the EP electrodes identified as 360-1 and 360-2, which serve as the reference and counter electrodes, extend along the length of the container 12. The working wire 100 has locations where sensors are to be formed, such as skived portions 1315 where insulation has been removed from the wire. Continuous operation may be implemented by feeding the continuous wire from spool 1310 into the container 12 (e.g., trough) in incremental steps, pausing for EP cycles to be performed on each section. For example, a length of wire corresponding to the length of the container 12 (e.g., a section of wire that contains 5-10 skived portions 1315 may be positioned in the container 12. Electropolymerization cycles are performed to build the desired total membrane thickness, and then the next section of wire is moved into the container area. The completed length of wire that has been electropolymerized may be rolled onto a storage spool 1320 or may be fed into a subsequent manufacturing step.

The container 12 may include a first end 1330 opposite a second end 1340, with both ends having concave top edges that allow the wire to easily access the electropolymerization solution 348 within the container. This configuration is illustrated in FIG. 13A. In other examples, the first end 1330 and the second end 1340 may be rectangular or another shape. FIG. 13B shows a longitudinal cross-sectional view of FIG. 13A, in accordance with some aspects. The container 12 is shown containing electropolymerization solution 348, where tension on the wire is relaxed slightly to allow the wire to be submerged in the electropolymerization solution 348.

FIG. 13C is a flowchart of a method for electropolymerizing a material onto a plurality of working wires for biological sensors in a continuous manner, in accordance with some aspects. The particular steps, order of steps, and combination of steps are shown for illustrative and explanatory purposes only. Other embodiments can implement different particular steps, orders of steps, and combinations of steps to achieve similar functions or results.

A method 1350 for electropolymerizing a material onto a plurality of working wires for biological sensors in a continuous manner may be performed, for example, using the systems described herein. At block 1360, a working wire is provided on a spool 1310 or other continuous wire source. The continuous wire from spool 1310 is feed into the container 12 which may be a trough or similarly elongated vessel containing the electropolymerization solution 348. At block 1370, one or more electropolymerization cycles are applied to the section of wire positioned within the container 12. During each cycle, voltage is applied across the working wire and the corresponding electrodes, and the resulting current is monitored to assess the progression of the reaction. At block 1380, the electropolymerization is considered complete when the current decreases to a minimal or near-zero level, indicating that the reaction has reached a self-limiting state. Additional cycles may be applied at this stage to ensure uniformity and full completion of the polymer layer. At block 1390, once the desired number of cycles has been completed for the current section of wire, the next section of wire from spool 1310 is advanced into the container 12 for processing.

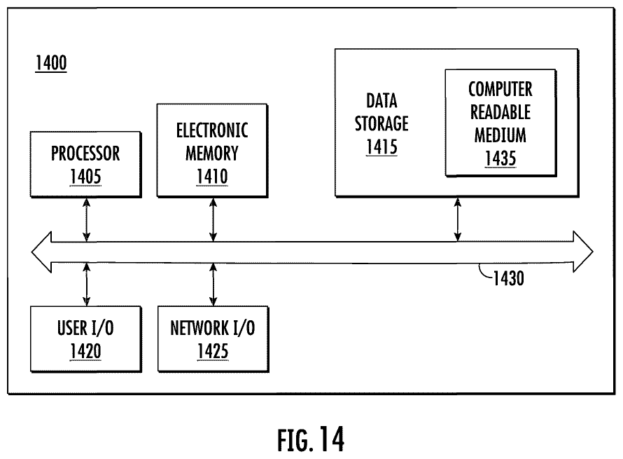

FIG. 14 is a simplified schematic diagram showing an example computer 1400 (representing any combination of one or more of the computers) which may be used to implement the controller 322 of the systems and methods described herein, in accordance with some aspects. Other examples may use other components and combinations of components. The controller 322 may be embodied in or include one or more computing devices, such as the example computer 1400, depending on the complexity and specific configuration of the system. For example, the computer 1400 may represent one or more physical computer devices or servers, such as web servers, rack-mounted computers, network storage devices, desktop computers, laptop/notebook computers, etc., depending on the complexity. In some aspects implemented at least partially in a cloud network potentially with data synchronized across multiple geolocations, the computer 1400 may be referred to as one or more cloud servers. In some aspects, the functions of the computer 1400 are enabled in a single computer device. In more complex implementations, some of the functions of the computing system are distributed across multiple computer devices, whether within a single server farm facility or multiple physical locations. In some aspects, the computer 1400 functions as a single virtual machine.

In the illustrated example, the computer 1400 generally includes at least one processor 1405, a main electronic memory 1410, a data storage 1415, a user input/output (I/O) 1420, and a network I/O 1425, among other components not shown for simplicity, connected or coupled together by a data communication subsystem 1430. A non-transitory computer readable medium 1435 includes instructions that, when executed by the processor 1405, cause the processor 1405 to perform operations including calculations and methods as described herein.

In accordance with the description herein, the various components of the system or method generally represent appropriate hardware and software components for providing the described resources and performing the described functions. The hardware generally includes any appropriate number and combination of computing devices, network communication devices, and peripheral components connected together, including various processors, computer memory (including transitory and non-transitory media), input/output devices, user interface devices, communication adapters, communication channels, etc. The software generally includes any appropriate number and combination of conventional and specially-developed software with computer-readable instructions stored by the computer memory in non-transitory computer-readable or machine-readable media and executed by the various processors to perform the functions described herein.

Any method (also referred to as a “process” or an “approach”) described or otherwise enabled by the disclosure herein may be implemented by hardware components (e.g., machines), software modules (e.g., stored in machine-readable media), or a combination thereof. By way of example, machines may include one or more computing device(s), processor(s), controller(s), integrated circuit(s), chip(s), system(s) on a chip, server(s), programmable logic device(s), field programmable gate array(s), electronic device(s), special purpose circuitry, and/or other suitable device(s) described herein or otherwise known in the art. One or more non-transitory machine-readable media embodying program instructions that, when executed by one or more machines, cause the one or more machines to perform or implement operations comprising the steps of any of the methods described herein are contemplated herein. As used herein, machine-readable media includes all forms of machine-readable media (e.g., one or more non-volatile or volatile storage media, removable or non-removable media, integrated circuit media, magnetic storage media, optical storage media, or any other storage media, including RAM, ROM, and EEPROM) that may be patented under the laws of the jurisdiction in which this application is filed, but does not include machine-readable media that cannot be patented under the laws of the jurisdiction in which this application is filed.

Systems that include one or more machines and one or more non-transitory machine-readable media are also contemplated herein. One or more machines that perform or implement, or are configured, operable, or adapted to perform or implement operations comprising the steps of any methods described herein are also contemplated herein. Method steps described herein may be order independent and can be performed in parallel or in an order different from that described if possible to do so. Different method steps described herein can be combined to form any number of methods, as would be understood by one of ordinary skill in the art. Any method step or feature disclosed herein may be omitted from a claim for any reason. Certain well-known structures and devices are not shown in figures to avoid obscuring the concepts of the present disclosure. When two things are “coupled to” each other, those two things may be directly connected together, or separated by one or more intervening things. Where no lines or intervening things connect two particular things, coupling of those things is contemplated in at least one example unless otherwise stated. Where an output of one thing and an input of another thing are coupled to each other, information sent from the output is received in its outputted form or a modified version thereof by the input even if the information passes through one or more intermediate things. Any known communication pathways and protocols may be used to transmit information (e.g., data, commands, signals, bits, symbols, chips, and the like) disclosed herein unless otherwise stated. The words comprise, comprising, include, including and the like are to be construed in an inclusive sense (i.e., not limited to) as opposed to an exclusive sense (i.e., consisting only of). Words using the singular or plural number also include the plural or singular number, respectively, unless otherwise stated. The word “or” and the word “and” as used in the Detailed Description cover any of the items and all of the items in a list unless otherwise stated. The words some, any and at least one refer to one or more. The terms may or can are used herein to indicate an example, not a requirement—e.g., a thing that may or can perform an operation, or may or can have a characteristic, need not perform that operation, or have that characteristic in each example, but that thing performs that operation or has that characteristic in at least one example. Unless an alternative approach is described, access to data from a source of data may be achieved using known techniques (e.g., requesting component requests the data from the source via a query or other known approach, the source searches for and locates the data, and the source collects and transmits the data to the requesting component, or other known techniques).

Reference has been made in detail to aspects of the disclosed invention, one or more examples of which have been illustrated in the accompanying figures. Each example has been provided by way of explanation of the present technology, not as a limitation of the present technology. In fact, while the specification has been described in detail with respect to specific examples of the invention, it will be appreciated that those skilled in the art, upon attaining an understanding of the foregoing, may readily conceive of alterations to, variations of, and equivalents to these examples. For instance, features illustrated or described as part of one example may be used with another example to yield a still further example. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only, and is not intended to limit the invention.

What is claimed is:

1. A system for electropolymerizing a material for biological sensors, the system comprising:
a wire-holding fixture configured to be received in an electropolymerization (EP) station of a set of EP stations, the wire-holding fixture comprising a plurality of terminals, each terminal electrically coupled to a respective working wire of a plurality of working wires, the plurality of working wires extending away from the wire-holding fixture;
a set of EP stations, each EP station configured to receive the wire-holding fixture;
a container configured to hold an electropolymerization solution, the container positioned beneath the EP station such that the plurality of working wires is immersible in the electropolymerization solution;
a board comprising a plurality of electrical contacts, each electrical contact configured to electrically engage a corresponding terminal of the plurality of terminals; and
a controller configured to apply a voltage to the plurality of working wires through the respective terminals and electrical contacts to initiate electropolymerization of the material from the electropolymerization solution to form a polymer layer on each working wire of the plurality of working wires.

2. The system of claim 1, further comprising:
a plate positioned in contact with the container, the plate configured to hold a plurality of containers.

3. The system of claim 2, wherein the controller is configured to control a temperature of the electropolymerization solution via a hotplate positioned beneath the plate and in communication with the controller.

4. The system of claim 1, further comprising:
a mechanism configured to move the board into and out of contact with the plurality of terminals on the wire-holding fixture.

5. The system of claim 1, further comprising:
a robot in communication with the controller, the robot configured to insert the wire-holding fixture into the EP station of the set of EP stations and transport the wire-holding fixture between a staging area and the EP station of the set of EP stations.

6. The system of claim 5, wherein each EP station has a set of electrodes configured to apply the voltage across to the wire-holding fixture positioned within the EP station.

7. The system of claim 1, wherein the plurality of electrical contacts comprises pogo pins.

8. The system of claim 1, further comprising:
an optical reader positioned to read an identifier code on the wire-holding fixture, the optical reader in communication with the controller, wherein the controller is configured to store data associated with the working wire associated with the identifier code.

9. The system of claim 1, wherein the EP stations in the set of EP stations are arranged in a linear array, and the system comprises a plurality of sets of EP stations, and wherein the board has a corresponding linear array of the electrical contacts.

10. The system of claim 1, wherein the controller is configured to record electropolymerization parameters for each working wire, the parameters comprising at least one of the voltage, a current, a cycle time, a cycle count, or a voltammetry profile.

11. The system of claim 1, wherein the wire-holding fixture is configured to hold four or more working wires.

12. A method for electropolymerizing a material for a biological sensor, the method comprising:
inserting a wire-holding fixture into an electropolymerization (EP) station of a set of EP stations, the wire-holding fixture configured to hold a plurality of working wires and comprising a plurality of terminals, each terminal of the plurality of terminals coupled to a respective working wire of the plurality of working wires, the plurality of working wires extending away from the wire-holding fixture;
positioning a container having an electropolymerization solution beneath the EP station of the set of EP stations;
immersing the plurality of working wires into the electropolymerization solution;
positioning a board comprising a plurality of electrical contacts in contact with the wire-holding fixture, each electrical contact of the plurality of electrical contacts configured to electrically engage with each terminal of the plurality of terminals; and
applying, by a controller, a voltage to the plurality of working wires through the respective terminals and electrical contacts to initiate electropolymerization of the material from the electropolymerization solution forming a polymer layer on each working wire of the plurality of working wires.

13. The method of claim 12, further comprising:
placing the container having with the electropolymerization solution on a plate configured to hold a plurality of containers.

14. The method of claim 13, further comprising:
adjusting the temperature of the plate using a hotplate positioned beneath the plate, the hotplate comprising a thermally conductive material and in communication with the controller, the controller configured to control the temperature of the electropolymerization solution via the hotplate.

15. The method of claim 12, further comprising:
actuating a mechanism to move the board into and out of contact with the plurality of terminals on the wire-holding fixture.

16. The method of claim 12, wherein the inserting of the wire-holding fixture into the EP station of the set of EP stations is by a robot in communication with the controller, the controller controlling a range of motion of the robot.

17. The method of claim 16, wherein each EP station includes a set of electrodes configured to apply the voltage across the wire-holding fixture positioned within the EP station.

18. The method of claim 12, wherein the plurality of electrical contacts comprises pogo pins.

19. The method of claim 12, further comprising:
reading, using an optical reader, an identifier code on the wire-holding fixture; and
storing, by the controller, manufacturing data associated with the working wire associated with the identifier code.

20. The method of claim 12, further comprising:
monitoring current while the applying of the voltage; and
repeating the applying of the voltage until the current decreases to a near-zero level.
